(19) United States
(12) Patent Application Publication
(10) Pub. No.: US 2016/0196383 A1
(43) Pub. Date: Jul. 7, 2016

(54) CLOUD SYSTEM FOR MENSTRUAL CYCLE PREDICTION AND METHOD THEREOF

(71) Applicant: YONGLIN BIOTECH CORP., New Taipei City (TW)

(72) Inventors: Jiann-Hua Wang, New Taipei City (TW); Chen-Yu Lin, New Taipei City (TW)

(21) Appl. No.: 14/972,469

(22) Filed: Dec. 17, 2015

Related U.S. Application Data
(60) Provisional application No. 62/099,746, filed on Jan. 5, 2015.

(30) Foreign Application Priority Data
Apr. 15, 2015 (TW) 104112105

(51) Int. Cl. G06F 19/28; A61B 10/00; G01N 33/493; G06N 5/02

(52) U.S. Cl. CPC G06F 19/28; G06N 5/02; A61B 10/0012; G01N 33/493

(57) ABSTRACT

Provided is a cloud system for menstrual cycle prediction, including a cloud server device, having an identity recognition unit, a memory unit, a transmitting module, a receiving module, a cloud database and an arithmetic processing unit; a test strip detection device, measuring a physiological hormone value of a plurality of test solutions from a user; and a portable electronic device, having a transmitting unit, a receiving unit, a display unit and a storing unit. The physiological hormone values of the plurality of test solutions that are measured by the test strip detection device are stored in the storing unit of the portable electronic device and are transmitted to the memory unit of the cloud server device, and the predicting of physiological menstrual cycle data of the user is performed by the arithmetic processing unit of the cloud server device.

### CROSS REFERENCE TO RELATED APPLICATIONS

This application claims priority benefits to the U.S. Provisional Patent Application No. 62/099,746 filed on Jan. 5, 2015 and the Taiwanese Patent Application No. 104112105 filed on Apr. 15, 2015, which are hereby incorporated by reference in their entirety.

### BACKGROUND OF THE INVENTION

1. Field of the Invention

The present invention relates to a cloud system and a method thereof, and relates particularly to a cloud system and a method thereof that can predict the menstrual cycle of women.

2. The Prior Arts

People of the modern day are often busy with work, and plans for starting a family are often bothersome to newly married young couples. In traditional Chinese societies, women face greater pressure compared to men when it comes to having children. As such, how women can effectively grasp the ovulation period which is key to pregnancy has become very important. Thus, newly married women will usually effectively grasp the ovulation date by means of using the ovulation agents or thermometers that are available in the market.

The ovulation agents are for measuring the changes in levels of the Follicle-Stimulating Hormone (FSH) and Luteinizing Hormone (LH). As shown in FIG. 1, the LH level of women will begin to increase before ovulation, reaches a peak on the day before ovulation, and the LH level will decrease rapidly after ovulation. The level of FSH reaches a high point seven days prior to ovulation, and begins to fall after two days. The level of FSH decreases and reaches a low point three days before ovulation, and will reach another high point on the day before ovulation. With regard to basal body temperature, the basal body temperature of women normally decreases by 0.1-0.2° C. prior to ovulation, and increases by 0.3-0.5° C. after ovulation. A temperature difference of 0.4-0.7° C. exists both before and after ovulation. The conventional ovulation agents predict the ovulation date of women by means of measuring level of FSH, level of LH and the basal body temperature.

However, since a lot of ovulation agents that are used in Taiwan are imported from Europe or the United States, therefore, the specifications of these ovulation agents comply with European or American specifications. The differences between the Europeans and Asians are therefore not considered. In terms of the ovulation period, there are differences in the values of FSH, LH and also values in the basal body temperature between European and American women as well as women from Asia. As such, if women from Asian countries directly use the ovulation agents that are imported from Europe or the United States, errors will always occur while using these ovulation agents.

In fact, following the advancement in technology, there has been a large increase in computing resources, storage resources as well as application resources. The traditional communications platform is converted to an intelligent arithmetic platform, enabling the users to access the services offered by the cloud system, by means of the browser or the smartphone apps. In the era of Internet of Things (IoT) and Big Data, if the Life Sciences and Information Technology can be combined, then personalized, predictable and preventable menstrual cycle data can be effectively provided to Asian women, and as such, a customized health management service with regard to the menstrual cycle can be provided to Asian women.

### SUMMARY OF THE INVENTION

In view of the drawbacks of the prior art, an objective of the present invention is to solve the problem of seeing measuring errors during the menstrual cycle of women, and also to solve the problem of not being able to effectively use the cloud system to provide personalized data on the menstrual cycle of women.

In order to achieve the above-mentioned objectives, the present invention provides a cloud system for menstrual cycle prediction, and includes a cloud server device, an identity recognition unit, a memory unit, a transmitting module, a receiving module, a cloud database and an arithmetic processing unit; a test strip detection device, for measuring the physiological hormonal value of a plurality of test solutions from a user; and a portable electronic device, having a transmitting unit, a receiving unit, a display unit and a storing unit, whereby the user information of the user may be registered into the identity recognition unit of the cloud server device, by means of the transmitting unit of the portable electronic device and the receiving module of the cloud server device. The physiological hormonal values of the plurality of test solutions measured by the test strip detection device may be stored in the storing unit of the portable electronic device, and may be transmitted to the memory unit of the cloud server device. The prediction of physiological menstrual cycle data of the user may be performed by the arithmetic processing unit of the cloud server device, based on the user information that may be identified by the identity recognition unit. The physiological hormonal values of the plurality of test solutions may be stored by the memory unit and the cloud database.

Preferably, the menstrual cycle data may include menstrual cycle prediction data, ovulation date prediction data and safe contraception date data, but is not limited to these.

Preferably, the physiological hormonal values of the plurality of test solutions may include a Follicle-Stimulating Hormone (FSH) value and a Luteinizing Hormone (LH) value, but is not limited to these.

Preferably, a date of safe contraception may be accurately predicted by means of computing the FSH value and the LH value and may be displayed on the portable electronic device.

Preferably, the user information may include a member registration, an age value and a basal body temperature value, but is not limited to these.

Preferably, after the arithmetic processing unit of the cloud server device finishes predicting the menstrual cycle data of the user, the portable electronic device may receive the menstrual cycle data of the user predicted by the cloud server device, by means of the receiving unit of the portable electronic device and the transmitting module of the cloud server device, and the menstrual cycle data of the user predicted by the cloud server device may be displayed on the portable electronic device.

Preferably, the plurality of test solutions may be urine from a human body, but is not limited to these.

Preferably, the identity recognition unit, the memory unit, the transmitting module, the receiving module and the arithmetic processing unit may be integrated into an integrated circuit.

Another objective of the present invention is to provide a cloud system prediction method for menstrual cycle of women, to solve the problem of measuring errors during the menstrual cycle, and also to solve the problem of not being able to effectively use the cloud system to provide personalized data on the menstrual cycle of women.

In order to achieve the above-mentioned objective, the cloud system prediction method for a menstrual cycle provided by the present includes the steps of: measuring a physiological hormonal value of a user from a plurality of test solutions by means of a test strip detecting device; registering user information of the user to an identity recognition unit of a cloud server device by means of a transmitting unit of a portable electronic device and a receiving module of the cloud server device; storing the physiological hormonal values of the plurality of test solutions measured by the test strip detecting device in a storing unit of the portable electronic device, and transmitting the physiological hormonal values of the plurality of test solutions measured by the test strip detecting device to a memory unit of the cloud server device, and predicting menstrual cycle data of the user by an arithmetic processing unit of the cloud server device in accordance with the user data identified by the identity recognition unit, the physiological hormonal values of the plurality of test solutions stored by the memory unit and a cloud database.

Preferably, the cloud system prediction method for menstrual cycle of women may also include the steps of receiving the menstrual cycle data of the user predicted by the cloud server device by means of a receiving unit of the portable electronic device and a transmitting module of the cloud server device, and displaying the menstrual cycle data of the user predicted by the cloud server device on the portable electronic device by means of a display unit of the portable electronic device.

Preferably, the menstrual cycle data may include the menstrual cycle prediction data, ovulation date prediction data and safe contraception date data.

Preferably, the cloud system prediction method for menstrual cycle of women may also include the steps of predicting a date of safe contraception by means of computing a Follicle-stimulating hormone (FSH) value and a luteinizing hormone (LH) value; and displaying the date of safe contraception on the portable electronic device.

In comparison with the prior art, the present invention provides an intelligent computing platform, by combining the computing resources, the storage resources and the application resources of the cloud server device. The users can access the services of the cloud service via the portable electronic device. The present invention effectively combines Life Science technology and Information technology to provide Asian women with a personalized, predictable and preventable menstrual cycle data, and as such, a customized health management service on the menstrual cycle can be provided to Asian women.

### BRIEF DESCRIPTION OF THE DRAWINGS

The present invention can be understood in more detail by reading the subsequent detailed description in conjunction with the examples and references made to the accompanying drawings, wherein:

FIG. 1 is a schematic diagram showing the conventional menstrual cycle of women.

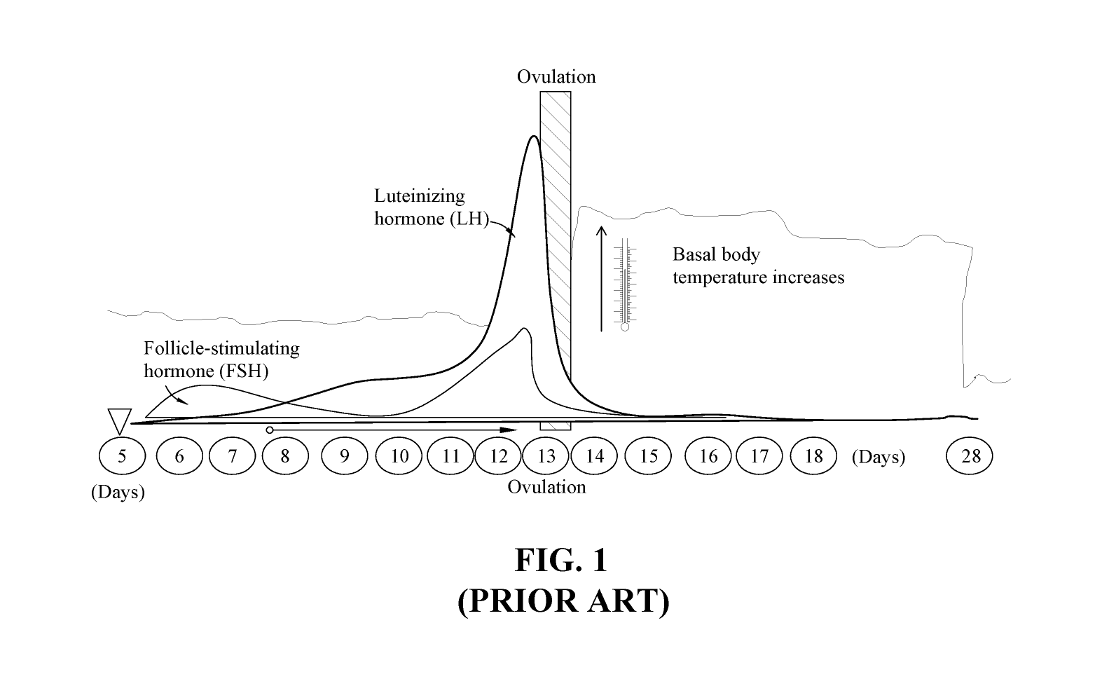

FIG. 2 is a general schematic diagram showing a cloud system for predicting the menstrual cycle of women, in accordance with an embodiment of the present invention.

FIG. 3 is a system block diagram of the cloud system for predicting the menstrual cycle of women, in accordance with an embodiment of the present invention.

FIG. 4 is a schematic diagram showing the menstrual cycle data of the cloud system for predicting the menstrual cycle of women, in accordance with an embodiment of the present invention.

FIG. 5 is a flow chart showing the menstrual cycle predicting method of the cloud system of the present invention.

### DETAILED DESCRIPTION OF THE PREFERRED EMBODIMENT

The present invention may be embodied in various forms and the details of the preferred embodiments of the present invention will be described in the subsequent content with reference to the accompanying drawings. The drawings (not to scale) depict only the preferred embodiments of the invention and shall not be considered as limitations to the scope of the present invention. Modifications of the shape of the present invention shall be considered within the spirit of the present invention.

It should be noted that the structures, proportions, sizes and the like of the drawings of the current specification are only for depicting the disclosures of the present invention, to enable easy reading and understanding by persons skilled in the art, and these are not meant to be conditions that limit the present invention. As such, the drawings are not technologically meaningful. Any modifications of the structure, proportion relationships or any adjustments of the sizes should fall within the scope that is covered by the technical content of the present invention, without affecting the effects produced by the present invention and without affecting the goals of the present invention.

According to the embodiments of the present invention, a cloud system for menstrual cycle prediction of the present invention is described as follows.

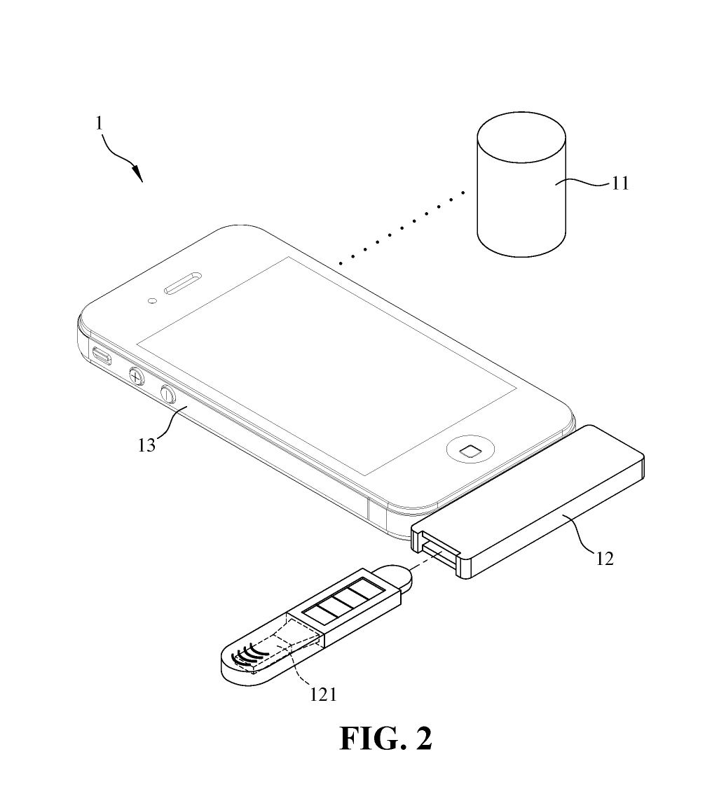

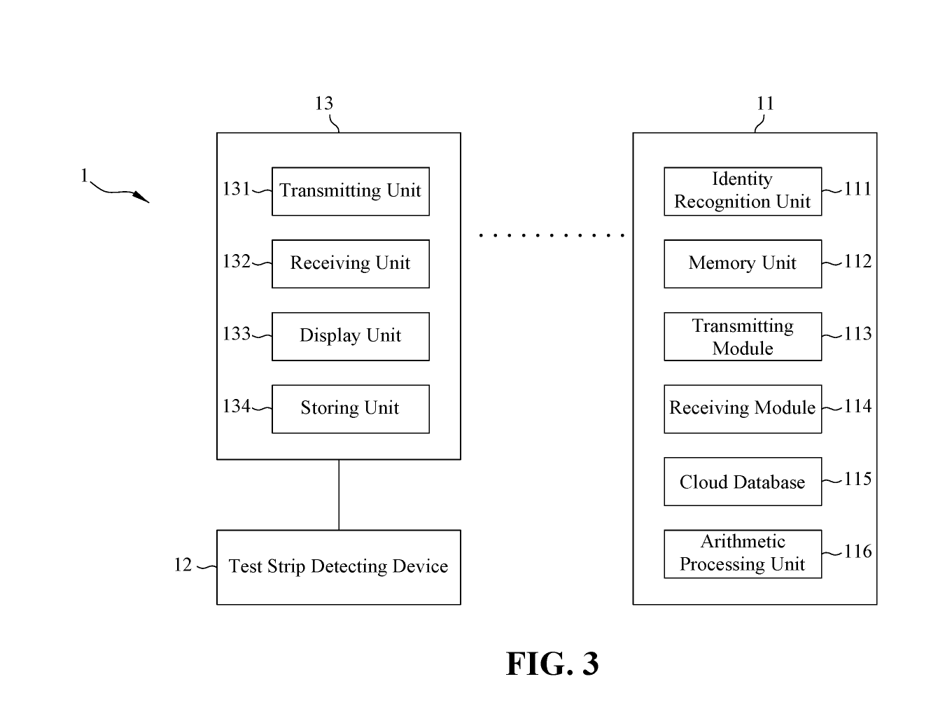

Please refer to FIGS. 2 and 3. FIG. 2 is a general schematic diagram of the present invention, and FIG. 3 is a system block diagram of the present invention. In accordance with a preferred embodiment of the present invention, the cloud system 1 for menstrual cycle prediction of the present invention includes: a cloud server device 11; whereby the cloud server device 11 may include an identity recognition unit 111, a memory unit 112, a transmitting module 113, a receiving module 114, a cloud database 115 and an arithmetic processing unit 116; a test strip detecting device 12; whereby the test strip detecting device 12 may include a test strip end 121, for measuring the physiological hormonal levels of the plurality of test solutions of a user (not shown in the drawings). The cloud server device may also include a portable electronic device 13, and the portable electronic device 13 may include a transmitting unit 131, a receiving unit 132, a display unit 133 and a storing unit 134, whereby the data from a user may be registered to the identity recognition unit 111 of the cloud server device 11, by means of the transmitting unit 131 of the portable electronic device 13 and the receiving module 114 of the cloud server device 11. Moreover, the physiological hormonal values of the plurality of test solutions measured by the test strip detecting device 12 may be stored in the storing unit 134 of the portable electronic device 13, and may also be transmitted to the memory unit 112 of the cloud server device 11; whereby the arithmetic processing unit 116 of the cloud server device 11 may predict the menstrual cycle data of the user in accordance with the user data identified by identity recognition unit 111, the physiological hormonal values of the plurality of test solutions stored in the memory unit 112 (for example, the physiological hormonal values for 5 consecutive days) and in accordance with the cloud database 115 stored in the memory unit 112.

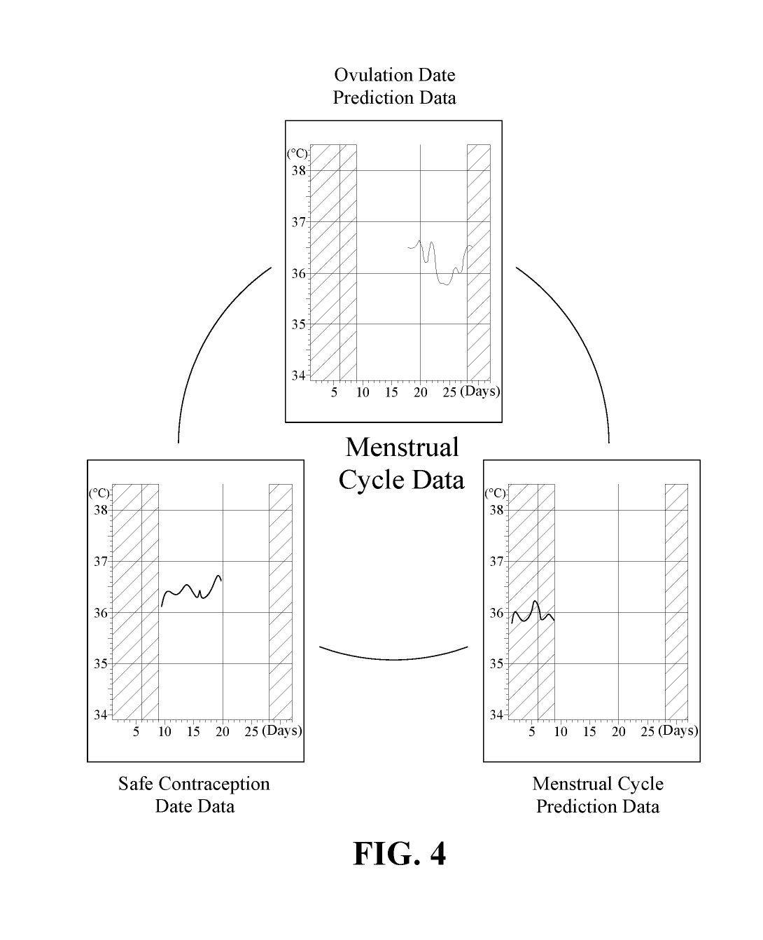

In accordance with a preferred embodiment of the present invention, the menstrual cycle data may include (a) menstrual cycle prediction data, an ovulation date prediction data, and a safe contraception date data. The above-mentioned data are shown in FIG. 4, but are not only limited to FIG. 4.

In accordance with a preferred embodiment of the present invention, the physiological hormonal values of the plurality of test solutions may include a Follicle-stimulating hormone (FSH) value and LH value, but is not limited to these.

In a preferred embodiment of the present invention, a date of safe contraception may be accurately predicted by means of computing the FSH value and the LH value and may be displayed on the portable electronic device.

In a preferred embodiment of the present invention, the user data may include a member registration, an age and a basal body temperature value, but is not limited to these.

In a preferred embodiment of the present invention, after the arithmetic processing unit 116 of the cloud server device 11 finishes predicting the menstrual cycle data of the user, the portable electronic device 13 may receive the menstrual cycle data of the user predicted by the cloud server device 11, by means of the receiving unit 132 of the portable electronic device 13 and the transmitting module 113 of the cloud server device 11. In addition, the menstrual cycle data of the user predicted by the cloud server device 11 may be displayed on the portable electronic device 13.

In a preferred embodiment of the present invention, the plurality of test solutions may be urine from a human body, but the present invention is not limited to this.

In a preferred embodiment of the present invention, the identity recognition unit 111, the memory unit 112, the transmitting module 113, the receiving module 114 and the arithmetic processing unit 116 may be integrated into an integrated circuit (not shown in the drawings), but the present invention is not limited to these.

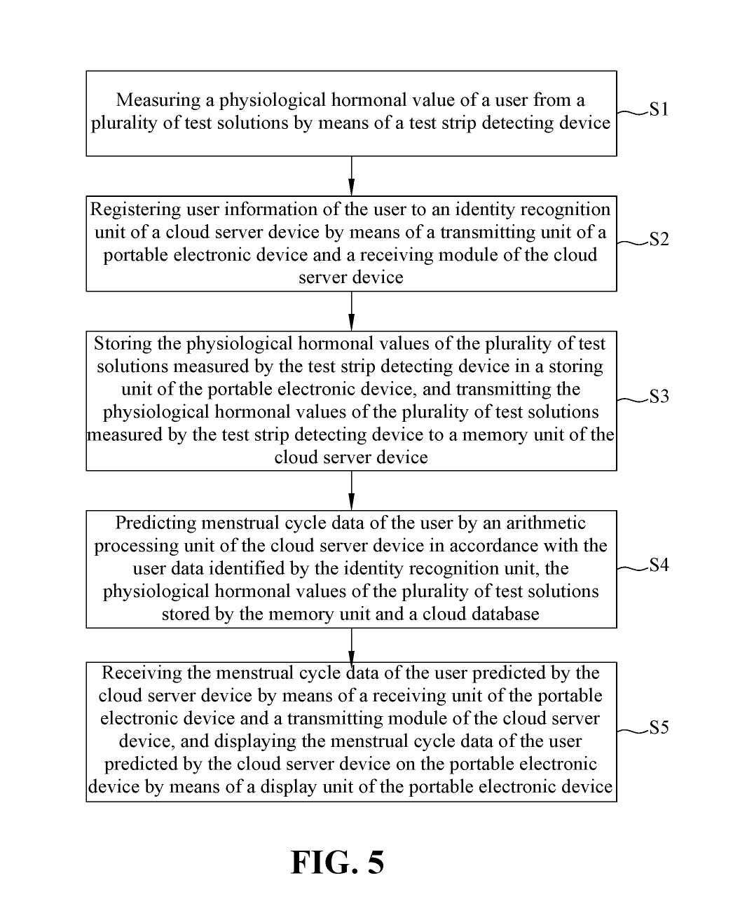

The present invention also provides a cloud system prediction method for the menstrual cycle, please refer to the flow chart as shown in FIG. 5. The cloud system prediction method for the menstrual cycle of the present invention includes the following steps: measuring the physiological hormonal values of the plurality of test solutions of the user by means of a test strip detecting device (Step S1); registering the user data of the user to an identity recognition unit of a cloud server device (Step S2); storing the physiological hormonal values of the plurality of test solutions measured by the test strip detecting device to a storing unit of the portable electronic device, as well as transmitting to a memory unit of the cloud server device (Step S3); and predicting the menstrual cycle data of the user by the operating processing unit of the cloud server device in accordance with the user information identified by the identity recognition unit, the physiological hormonal values of the plurality of test solutions stored by the memory unit and the cloud database (Step S4).

The method of the present invention may include the step of the portable electronic device receiving the menstrual cycle data of the user predicted by the cloud server device, by means of the receiving unit of the portable electronic device and the transmitting module of the cloud server device. In addition, the method of the present invention may also include displaying the menstrual cycle data of the user predicted by the cloud server device on the portable electronic device, by means of a display unit of the portable electronic device (Step S5).

In the method of the present invention, the menstrual cycle data may include menstrual cycle prediction data, ovulation prediction data and a safe contraception date data, but is not limited to these.

Also, the method of the present invention may include the steps of predicting a date of safe contraception by means of computing a Follicle-stimulating hormone (FSH) value and a luteinizing hormone (LH) value, and displaying the date of safe contraception on the portable electronic device.

In comparison with the prior art, the present invention provides an intelligent arithmetic platform, by combining the arithmetic resources of the cloud server device, the storing resources and the application resources. The users may access the services on the cloud system via the portable electronic device. In addition, the present invention provides Asian women with a personalized, predictable and preventable data on their menstrual cycle, by effectively combining the Life Science technologies and Information technologies. As such, a customized menstrual cycle health management service is provided to Asian women.

Although the present invention has been described with reference to the preferred embodiments, it will be understood that the invention is not limited to the details described thereof. Various substitutions and modifications have been suggested in the foregoing description, and others will occur to those of ordinary skill in the art. Therefore, all such substitutions and modifications are intended to be embraced within the scope of the invention as defined in the appended claims.

What is claimed is:

1. A cloud system for menstrual cycle prediction, comprising:
a cloud server device, having an identity recognition unit, a memory unit, a transmitting module, a receiving module, a cloud database and an arithmetic processing unit;
a test strip detection device, measuring a physiological hormone value of a plurality of test solutions from a user; and
a portable electronic device, having a transmitting unit, a receiving unit, a display unit and a storing unit, wherein user information of the user is registered into the identity recognition unit of the cloud server device, by means of the transmitting unit of the portable electronic device and the receiving module of the cloud server device, and the physiological hormone values of the plurality of test solutions that are measured by the test strip detection device are stored in the storing unit of the portable electronic device and are transmitted to the memory unit of the cloud server device,
wherein the predicting of physiological menstrual cycle data of the user is performed by the arithmetic processing unit of the cloud server device, based on the user information that is identified by the identity recognition unit, the physiological hormonal values of the plurality of test solutions that are stored by the memory unit and the cloud database.

2. The cloud system for menstrual cycle prediction of claim 1, wherein the menstrual cycle data comprises menstrual cycle prediction data, ovulation date prediction data and a safe contraception date data.

3. The cloud system for menstrual cycle prediction of claim 1, wherein the physiological hormonal values of the plurality of test solutions comprise a Follicle-stimulating hormone (FSH) value and a luteinizing hormone (LH) value.

4. The cloud system for menstrual cycle prediction of claim 3, wherein a date of safe contraception is accurately predicted by means of computing the FSH value and the LH value and is displayed on the portable electronic device.

5. The cloud system for menstrual cycle prediction of claim 1, wherein the user information comprises a member registration, an age value and a basal body temperature value.

6. The cloud system for menstrual cycle prediction of claim 1, wherein after the arithmetic processing unit of the cloud server device finishes predicting the menstrual cycle data of the user, the portable electronic device receives the menstrual cycle data of the user predicted by the cloud server device, by means of the receiving unit of the portable electronic device and the transmitting module of the cloud server device, and the menstrual cycle data of the user predicted by the cloud server device are displayed on the portable electronic device.

7. The cloud system for menstrual cycle prediction of claim 1, wherein the plurality of test solutions is urine from a human body.

8. The cloud system for menstrual cycle prediction of claim 1, wherein the identity recognition unit, the memory unit, the transmitting module, the receiving module and the arithmetic processing unit are integrated into an integrated circuit.

9. A cloud system prediction method for a menstrual cycle, comprising the steps of:
measuring a physiological hormonal value of a user from a plurality of test solutions by means of a test strip detecting device;
registering user information of the user to an identity recognition unit of a cloud server device by means of a transmitting unit of a portable electronic device and a receiving module of the cloud server device;
storing the physiological hormonal values of the plurality of test solutions measured by the test strip detecting device in a storing unit of the portable electronic device, and transmitting the physiological hormonal values of the plurality of test solutions measured by the test strip detecting device to a memory unit of the cloud server device, and
predicting menstrual cycle data of the user by an arithmetic processing unit of the cloud server device in accordance with the user data identified by the identity recognition unit, the physiological hormonal values of the plurality of test solutions stored by the memory unit and a cloud database.

10. The cloud system prediction method for a menstrual cycle of claim 9, further comprising the steps of receiving the menstrual cycle data of the user predicted by the cloud server device by means of a receiving unit of the portable electronic device and a transmitting module of the cloud server device, and displaying the menstrual cycle data of the user predicted by the cloud server device on the portable electronic device by means of a display unit of the portable electronic device.

11. The cloud system prediction method for a menstrual cycle of claim 10, wherein the menstrual cycle data comprises menstrual cycle prediction data, ovulation date prediction data and safe contraception date data.

12. The cloud system prediction method for a menstrual cycle of claim 9, further comprising the steps of predicting a date of safe contraception by means of computing a Follicle-stimulating hormone (FSH) value and a luteinizing hormone (LH) value; and displaying the date of safe contraception on the portable electronic device.
